(19) United States
(12) Patent Application Publication
(10) Pub. No.: US 2026/0355455 A1
(43) Pub. Date: Nov. 12, 2026

(54) Closed-loop Biofeedback System and Method for Synchronized Auditory and Visual Stimulation to Enhance Sleep Quality and Neurological Health

(71) Applicant: Michael Larson, Colorado Springs, CO (US)

(72) Inventor: Michael Larson, Colorado Springs, CO (US)

(21) Appl. No.: 19/672,686

(22) Filed: May 10, 2026

Related U.S. Application Data
(60) Provisional application No. 63/803,821, filed on May 11, 2025.

(51) Int. Cl. A61M 21/02; A61M 21/00; A61N 5/06

(52) U.S. Cl. CPC A61M 21/02; A61N 5/0622; A61M 2021/0027; A61M 2021/0044; A61M 2205/3303; A61M 2205/3327; A61M 2205/3584; A61M 2205/50; A61M 2205/8206; A61M 2209/088; A61M 2210/0612; A61M 2210/0662; A61M 2230/005; A61M 2230/10; A61M 2230/14; A61M 2230/63; A61N 2005/0627; A61N 2005/0648; A61N 2005/0652

(57) ABSTRACT

A closed-loop wearable system enhances sleep quality and neurological health by delivering synchronized binaural auditory tones and red-light photomodulation (620-650 nm) under real-time EEG/EOG/IMU feedback. On-device processing classifies sleep stage and dynamically adjusts stimulation frequencies to accelerate deep-sleep onset, deepen slow-wave activity, preserve REM microarchitecture, and promote 40 Hz-mediated glymphatic clearance. Optional embodiments include headband-integrated or modular configurations with machine-learning personalization.

### CROSS REFERENCE TO RELATED APPLICATIONS

This application claims priority under 35 U.S.C. § 119(e) to U.S. Provisional Patent Application No. 63/803,821, filed May 11 , 2025, entitled “Synchronized Audio and Visual Stimulation to Enhance Sleep Quality,” the entire disclosure of which is hereby incorporated by reference in its entirety.

### FIELD OF THE INVENTION

The present invention relates generally to wearable neurostimulation devices and, more particularly, to closed-loop biofeedback systems that utilize real-time electroencephalography (EEG), electrooculography (EOG), and motion sensing to deliver synchronized binaural auditory tones and red-light photomodulation for brainwave entrainment, sleep-stage optimization, glymphatic clearance enhancement, and neurological health support.

### BACKGROUND OF THE INVENTION

Sleep disorders affect tens of millions of individuals and are associated with impaired cognitive performance, increased risk of neurodegenerative disease, and diminished quality of life. Conventional pharmacological interventions frequently disrupt natural sleep architecture, suppress slow-wave sleep (SWS), or produce undesirable side effects such as sleep inertia and dependence. Passive audio or light devices lack real-time adaptation to the user's dynamic brain state and fail to integrate multimodal stimuli in a closed-loop manner.

While certain prior systems have employed EEG-based feedback to deliver binaural tones or other auditory stimuli (see, e.g., U.S. Pat. Nos. 9,764,110; 9,987,459; 11,986,649; 11,172,859; and 11,458,279), none teach or suggest the novel combination of real-time synchronized auditory binaural beats and red-light photomodulation (620-650 nm) under closed-loop control that further incorporates EOG and IMU data for precise sleep-stage classification, dynamically embeds a 40 Hz carrier/flicker component during N3 and REM to promote glymphatic clearance, and employs machine-learning personalization across multiple sleep sessions. The present invention solves the long-felt need for a multimodal, stage-aware, non-pharmacological system that simultaneously accelerates sleep onset, deepens slow-wave sleep, preserves REM microarchitecture, and provides neurological housekeeping benefits not achieved by single-modality or open-loop devices.

Research has clearly shown that specific brainwave frequency states are associated with significant physiological benefits. In particular, deep sleep characterized by slow waves is linked to restorative processes, while both slow-wave sleep (SWS) and 40 Hz stimulation promote glymphatic brain cleaning. The 40 Hz frequency has also been associated with potential benefits in connection with dementia. Studies have further confirmed that particular brainwave states can be reinforced and encouraged through brain entrainment using sensory stimulation, especially auditory and visual stimulation, and that these modalities are more effective when applied in concert. The present invention employs natural stimulation to boost particular brainwave states and thereby realize the greater benefits associated with those states.

Existing brainwave entrainment technologies, while promising, suffer from open-loop operation, lack of personalization, and insufficient integration of auditory binaural beats with visual photomodulation at frequencies shown to promote glymphatic waste clearance (e.g., 40 Hz gamma stimulation). A need therefore exists for a comfortable, non-invasive, wearable system that (i) continuously monitors polysomnographic signals, (ii) classifies sleep macro-and micro-architecture in real time, and (iii) automatically adjusts synchronized auditory and visual stimuli to accelerate sleep onset, deepen slow-wave sleep, preserve REM microarchitecture, and potentiate neurological housekeeping mechanisms without pharmacological intervention.

### SUMMARY OF THE INVENTION

The present invention satisfies the foregoing need by providing a closed-loop biofeedback system that integrates real-time EEG/EOG/inertial measurement unit (IMU) sensing with on-device signal processing to deliver precisely synchronized binaural auditory stimulation (BNB) and red-light photomodulation (PM). In preferred embodiments, the system comprises a lightweight fabric headband containing a single dry forehead EEG electrode and a single EOG electrode positioned near one eye (with optional additional electrodes of both types for enhanced robustness and local artifact identification), an inertial measurement unit (IMU), comfortable wired earbuds connected by wires to the on-board microprocessor, red LEDs (approximately 620-650 nm) housed in an integral eye mask, a microcontroller, and a rechargeable battery.

The processor continuously computes an effective brainwave frequency via Fast Fourier Transform (FFT) and classifies sleep stage (Wake, N1-N3, REM) using a low-power machine-learning model specifically engineered to solve the real-time “one-direction” (online) problem. The system dynamically adjusts a 40 Hz carrier tone and a variable binaural beat frequency, together with synchronized PWM-controlled red-light pulsing, to entrain brainwave activity toward therapeutically beneficial states according to the multi-mode protocol described herein. Machine-learning models refine stimulation parameters over time using logged data. Optional embodiments include standalone auditory or visual modes, adhesive electrode configurations, and wireless earbuds.

The invention thereby provides faster sleep onset, increased N3 duration, improved cognitive performance upon awakening, and long-term neurological benefits without the drawbacks of hypnotics or stimulants.

### BRIEF DESCRIPTION OF THE DRAWINGS

FIG. 1 is an external perspective view of the preferred embodiment of the closed-loop biofeedback sleep enhancement system 10 being worn by a user, showing the fabric headband 20, wired earbuds 60, wires connecting wired earbuds to the headband 65, and eye mask 70.

FIG. 2 is a view of the interior (side facing a user's head) of the preferred embodiment of the fabric headband 20 showing component placement, including the forehead dry EEG electrode 30, EOG electrode 50, ground/reference electrodes 55, wired earbuds 60, connecting earbud wires 65, eyemask portion of the headband 70, padded eye cup 74, diffusing surface of the raised portion of the eye cup 75, red LEDs 80, USB-C charging port 130, and hook and loop fastening pads 95. Revealed in cut-away views, with the inner layer of fabric peeled back, are electronic components located between inner and outer fabric layers including the inertial measurement unit 90, electronics module 100, microcontroller 110, rechargeable battery 120, and Bluetooth transceiver module 140.

FIG. 3A is a graph illustrating an audible waveform 202, possessing a carrier tone period 201 which results from exposure to two pure sinusoidal tones of differing frequencies through left and right stereo channels, and the corresponding perceived binaural beat envelope 203 possessing a period 200.

FIG. 3B is a graph illustrating an intensity-modulated visual stimulus waveform synchronized to the audible stimulus of FIG. 3A. Shown are the period of the perceived pulsing light 210, period of the light flicker 211, flicker waveform 212, and the envelope of the waveform that creates the sensation of pulsing 213).

FIG. 3C is a graph illustrating a pulse-width-modulated (PWM) visual stimulus waveform synchronized to the audible stimulus of FIG. 3A. Shown are the period of the perceived pulsing light 220, period of the light flicker 221, flicker (PWM) waveform 222, and the envelope of the waveform that creates the sensation of pulsing 223).

FIG. 4 is a high-level block diagram illustrating the major components and closed-loop data flow of the system. Included are the data gathering sensors: forehead dry EEG electrode 30, EOG electrode 50, and the IMU motion detector 90. Data measurements from these feed into the processor 110, which creates and delivers the stimuli through the wired earbuds 60 and the LEDs 80. The physiological responses to the stimuli are then measured by the sensors in the closed loop system design. The rechargeable battery 120 powers the processor 110. The Bluetooth module 140 communicates with an external smart device and receives setting and data information for the processor 110, and transmits data out to an external smart device.

FIG. 5 is a flowchart depicting the real-time closed-loop control algorithm, including signal acquisition, FFT processing, sleep-stage classification, and dynamic stimulus adjustment. The steps shown include: power on and control algorithm initiation 500, hardware initialization and raw sensor data epoch acquisition/preprocessing 510, data quality criterion decision 520, feature extraction and sleep stage classification 530, classifier confidence threshold decision 540, mode dispatch decision 550, onset descent mode stimulus parameters 560, N2 descent mode stimulus parameters 570, N3 slow wave sleep mode stimulus parameters 580, REM mode stimulus parameters 590, cumulative N3 duration check 610, epoch data logging and timer update 620, stimulus amplitude ramp-down and fade-out 630, arousal or undesired stage transition detection decision 640, prepare for next epoch 650, and session termination and power down 660.

(The drawings are illustrative and not drawn to scale. Various components may be rearranged, omitted, or added without departing from the scope of the invention.)

### DETAILED DESCRIPTION OF THE INVENTION

The following description is presented to enable any person skilled in the art to make and use the invention. It is provided in the context of particular embodiments and is not intended to be limiting. Various modifications will be readily apparent to those skilled in the art, and the generic principles defined herein may be applied to other embodiments without departing from the spirit or scope of the invention.

Reference numerals appearing in the drawings and description correspond to like elements. The terminology used is for the purpose of describing particular embodiments only and is not intended to be limiting.

The present invention solves the long-felt need for a multimodal, stage-aware, non-pharmacological system that simultaneously accelerates sleep onset, deepens slow-wave sleep, preserves REM microarchitecture, and provides neurological housekeeping benefits not achieved by single-modality or open-loop devices.

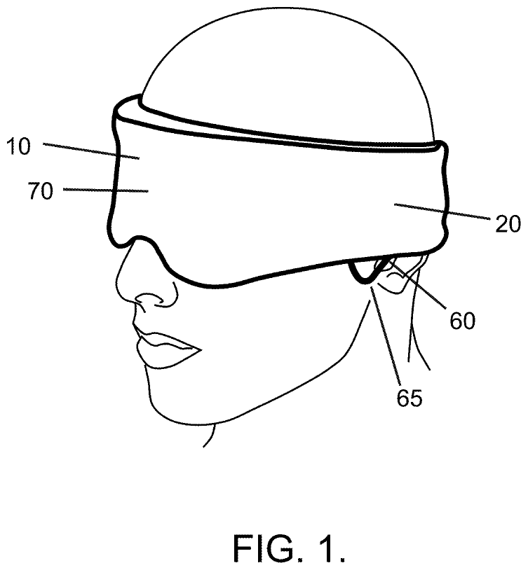

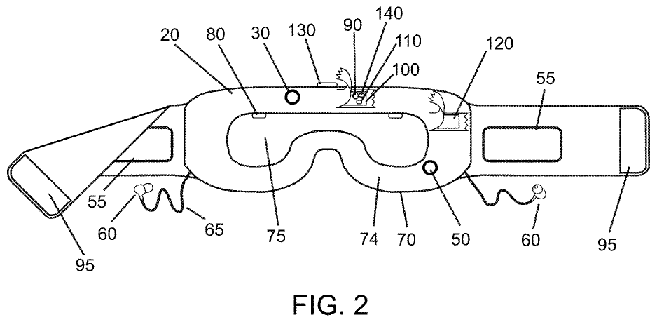

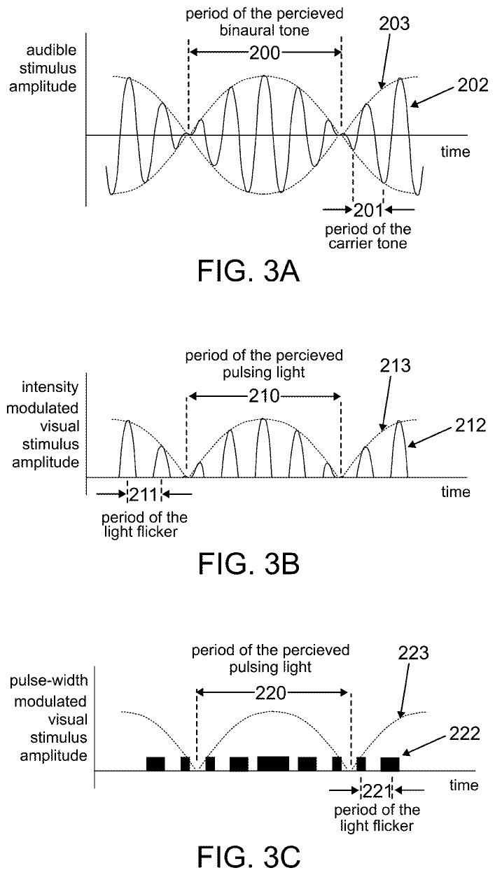

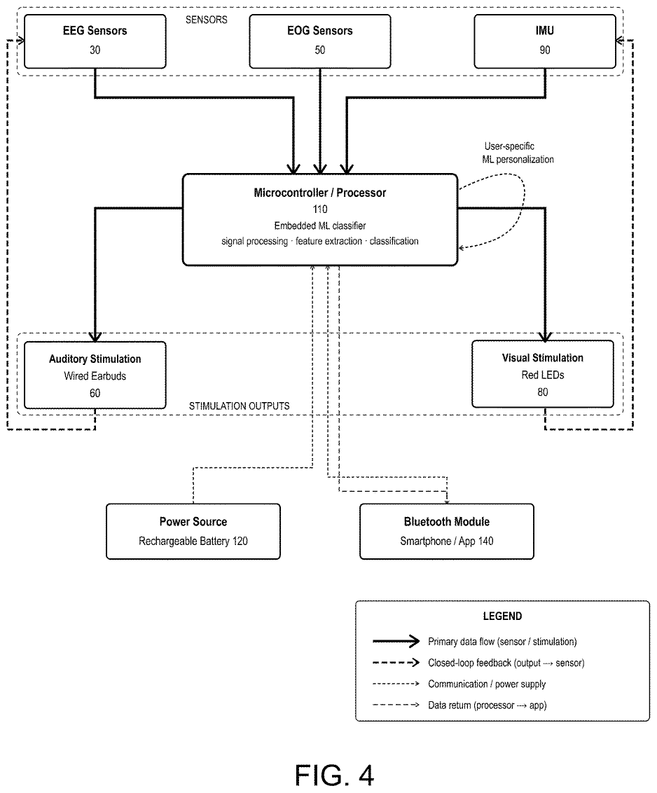

### Preferred Embodiment—Headband Configuration (shown in FIG. 1 and FIG. 2)

The closed-loop biofeedback sleep enhancement system 10 is embodied as a comfortable, breathable fabric headband 20 (e.g., hypoallergenic cotton-polyester blend) that is worn across the eyes and forehead and around the back of the head. Integrated components include:
A single dry forehead EEG electrode 30 (e.g., conductive-polymer or metallic) for primary brainwave acquisition. In certain embodiments, one or more additional EEG electrodes may be included.
A single EOG electrode 50 (peri-ocular) positioned near one eye (approximately 1 cm lateral and 1 cm inferior to the outer canthus) to detect eye movements. In certain embodiments, bilateral or multiple EOG electrodes are used for more robust detection of horizontal and vertical eye movements and for improved local artifact identification. In certain embodiments a single EOG electrode 50 positioned near one eye may be referenced to either a ground/reference electrode 55 (located one at each ear) or to the forehead EEG electrode 30 to derive a usable electrooculographic signal for sleep-stage classification and REM detection.
Ground/reference electrode 55 (located one at each ear) made using conductive fabric and integral with the headband for sleeping comfort.
Inertial measurement unit (IMU)/accelerometer 90 configured to measure acceleration, including all or part of linear and rotational measures about three or fewer axes, to confirm REM sleep via detection of muscle atonia and reduced movement.
Comfortable wired earbuds 60 connected by connecting wires to earbuds 65 to the on-board microcontroller/microprossor 110 and positioned to fit comfortably under the headband for delivery of binaural tones directly into the ear canal.
An integral eye mask 70 containing two red LEDs for photomodulation stimulation 80 positioned to illuminate eyes through closed eyelids. The eye mask further includes a padded eye cup 74 (to create a space for illumination around both eyes) lined with a diffusing surface 75 (lining the eye cup for uniform illumination around both eyes).
Low-power microcontroller/microprocessor 110 with on-board FFT, digital filtering, TensorFlow Lite Micro, and other capabilities.
300-500 mAh rechargeable battery 120 supporting ≥8 hours of operation, rechargeable via USB-C charging port 130.
Bluetooth transceiver module 140 for optional smartphone app connectivity.
Hook and loop fastening pads 95 to fasten the back of the headband on the user's head in an adjustable manner.
Electronics module 100 (located between fabric layers) containing the microcontroller/microprocessor 110, rechargeable battery 120, USB-C charging port 130, and Bluetooth transceiver module 140.

(The interior placement of the electronics module between fabric layers is illustrated in the cut-away, peel-back portions around the padded eye cup 74 in FIG. 2.)

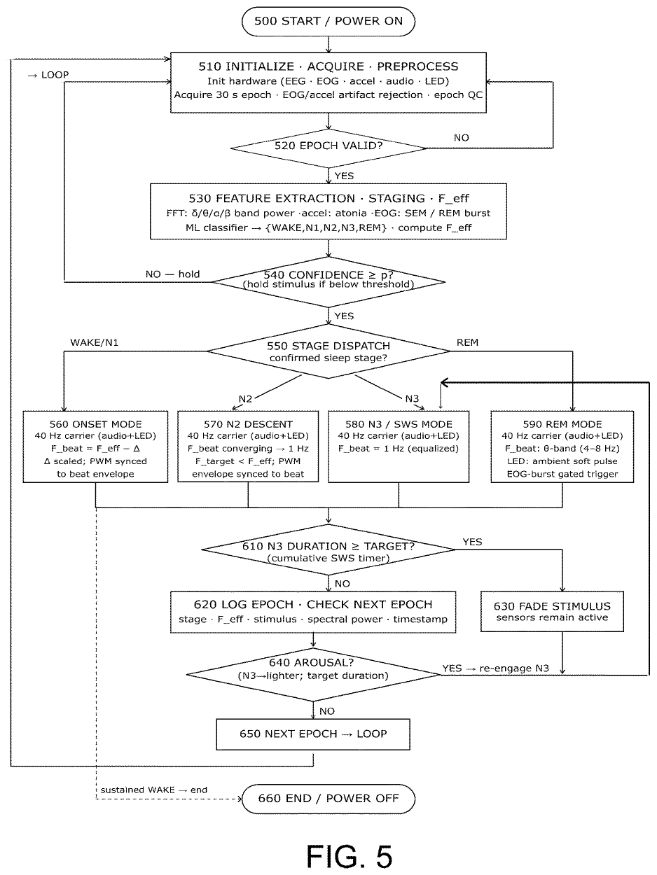

### Closed-Loop Control Algorithm (FIG. 5)

The real-time closed-loop sleep entrainment control algorithm of the present invention is illustrated in FIG. 5. The algorithm executes continuously on the onboard processor of the headband device and governs all aspects of sensor acquisition, sleep stage classification, and adaptive stimulus delivery throughout a sleep session.

At step (500), the device is powered on and the control algorithm is initiated. At step (510), the processor initializes all hardware subsystems, including the EEG sensor, the EOG sensor, the three-axis accelerometer, the audio output drivers coupled to the stereo earbuds, and the LED drivers coupled to the diffused red LEDs. Following initialization, step (510) further acquires a raw sensor data epoch of a programmable duration, defaulting to thirty seconds in accordance with American Academy of Sleep Medicine (AASM) convention, comprising EEG, EOG, and accelerometer signals. Step (510) further applies artifact rejection processing to the acquired epoch, including amplitude-threshold-based rejection, EOG-correlation-based ocular artifact subtraction, and accelerometer-based motion gating, yielding a preprocessed epoch candidate.

At decision step (520), the processor evaluates whether the preprocessed epoch satisfies a minimum data quality criterion. If the epoch does not satisfy the quality criterion, indicating excessive artifact content, control returns to the acquisition portion of step (510) and a new epoch is acquired. If the epoch satisfies the quality criterion, control proceeds to step (530).

At step (530), the processor performs multi-domain feature extraction and sleep stage classification. Feature extraction comprises Fast Fourier Transform (FFT) spectral analysis of the EEG signal to compute absolute and relative band power in the delta (0.5-4 Hz), theta (4-8 Hz), alpha (8-13 Hz), and beta (13-30 Hz) bands, together with the power spectrum. Step (530) further extracts oculomotor features from the EOG signal, including detection of slow eye movements characteristic of sleep onset and rapid eye movement (REM) burst events characteristic of REM sleep, and extracts an atonia index. The extracted features are supplied to a machine learning classifier, e.g., a hybrid hidden Markov model and neural network architecture trained on one or more publicly available polysomnography databases, which outputs a sleep stage classification drawn from the set {WAKE, N1, N2, N3, REM} together with an associated confidence value p. Step (530) further computes the effective brainwave frequency F_eff as a smoothed, weighted spectral centroid derived from the EEG power spectrum over a rolling epoch window.

At decision step (540), the processor compares the confidence value p output by the classifier of step (530) against a minimum confidence threshold p_min. If p is less than p_min, the current stimulus parameters are held unchanged and control returns to step (510) to acquire a subsequent epoch, thereby preventing spurious stimulus transitions arising from uncertain classifications. If p is greater than or equal to p_min, control proceeds to step (550).

At decision step (550), the processor dispatches control to one of four mode-specific stimulus parameter blocks based on the confirmed sleep stage. If the confirmed stage is WAKE or N1, control proceeds to step (560). If the confirmed stage is N2, control proceeds to step (570). If the confirmed stage is N3, control proceeds to step (580). If the confirmed stage is REM, control proceeds to step (590).

At step (560), the onset mode stimulus parameters are established. The audio output is configured as a binaural beat stimulus comprising a 40 Hz carrier, wherein a first pure sinusoidal tone at frequency f_L is delivered to the left earbud and a second pure sinusoidal tone at frequency f_R is delivered to the right earbud, with the beat frequency F_beat equal to the arithmetic difference between f_L and f_R. The beat frequency F_beat is set to a target frequency F_target computed as F_eff minus an offset Δ, wherein Δ is scaled as a function of the current stage depth to position F_target below the brain's current effective frequency, thereby inducing a frequency-following response that leads the brain toward lower oscillatory states. The LED visual stimulus is configured to pulse in perceived intensity at a rate synchronized to F_beat, achieved through pulse-width modulation (PWM) of the 40 Hz LED flicker carrier.

At step (570), the N2 descent mode stimulus parameters are established. The 40 Hz binaural carrier and phase-locked LED configuration of step (560) are maintained. The beat frequency F_target is continuously updated to converge toward 1 Hz as F_eff decreases, maintaining a leading offset below F_eff such that the target and effective frequencies equalize at 1 Hz upon entry into N3 sleep. Stimulus amplitude is progressively reduced as staging confidence in N3 increases.

At step (580), the N3 slow wave sleep mode stimulus parameters are established. The 40 Hz binaural carrier and phase-locked LED configuration are maintained. The beat frequency is set to 1 Hz, corresponding to the slow oscillation frequency of N3 sleep. Step (580) additionally implements a slow oscillation phase-locked stimulation sub-protocol wherein the EEG signal is bandpass filtered at 0.5-1.5 Hz in real time and the positive peak of each detected slow oscillation cycle, corresponding to the cortical up-state, is identified. The LED PWM envelope is similarly synchronized to the slow oscillation phase.

At step (590), the REM mode stimulus parameters are established. The 40 Hz binaural carrier and LED flicker are maintained, consistent with continuous gamma-frequency therapeutic entrainment throughout the sleep session. The beat frequency is shifted to the theta band, preferably in the range of 4 to 8 Hz, to provide resonant reinforcement of the endogenous hippocampal-frontal theta rhythm characteristic of REM sleep. The LED visual stimulus is reconfigured to a softer ambient intensity modulation rather than a rigidly periodic pulse, so as not to impose a conflicting periodic rhythm upon the more labile REM brain state. Additionally, REM saccade burst events detected by the EOG sensor are used as trigger signals to deliver brief, phase-locked LED pulses synchronized to detected rapid eye movement onset, providing closed-loop visual stimulation tied to the brain's own REM activity.

At decision step (610), the processor evaluates whether the cumulative duration of confirmed N3 sleep during the current session has met or exceeded a programmable target duration. If the target duration has not been reached, control proceeds to step (620). If the target duration has been reached, control proceeds to step (630), at which the stimulus amplitude is ramped down and the auditory and visual stimuli are faded out, while the sensors continue to acquire and process data.

At step (620), the processor logs the epoch data record, comprising the confirmed stage, F_eff, the applied stimulus parameters, the EEG band power values, and the epoch timestamp, and advances the epoch counter and the cumulative SWS duration timer as appropriate. Control proceeds to decision step (640).

At decision step (640), the processor evaluates whether an arousal event or an undesired stage transition—specifically, a transition from N3 to a lighter sleep stage prior to achievement of the target SWS duration—has been detected. If such an event is detected, control proceeds directly to step (580) via the re-engagement path, bypassing the stage dispatch decision step (550), to reinstate N3-targeted stimulation without delay. If no arousal or drift is detected, control proceeds to step (650).

At step (650), the processor prepares for the subsequent epoch and control returns to step (510) for continued sensor acquisition and processing, thereby completing one iteration of the closed-loop control cycle.

If the confirmed sleep stage classification at step (550) or step (560) indicates sustained wakefulness exceeding a programmable timeout duration, the algorithm proceeds via the session termination path, indicated by a dashed line in FIG. 5, to step (660), at which the device concludes the sleep session and powers down.

### Materials and Implementation Details

Electrodes utilize low-impedance dry or gel-free contacts (20-50 kΩ). Electronics are encapsulated in flexible PCB housed within fabric folds. Firmware is written in C for deterministic real-time performance. The system is designed to meet general wellness device safety standards and is free of gels, heat, or mechanical forces that could disturb sleep.

The foregoing description of the preferred embodiments is not intended to be exhaustive or to limit the invention to the precise forms disclosed. Many modifications and variations are possible in light of the above teachings. It is intended that the scope of the invention be limited not by this detailed description, but rather by the claims appended hereto.

This specification discloses the invention in sufficient detail to enable one of ordinary skill in the art to make and use the claimed subject matter without undue experimentation. All patents, patent applications, and publications cited herein are hereby incorporated by reference in their entireties.

What is claimed is:

1. A closed-loop biofeedback system for enhancing sleep quality, comprising:
at least one sensor configured to measure real-time brain activity of a user;
a processor configured to determine a brain state or sleep stage of the user based on the measured brain activity;
an auditory stimulation device configured to deliver auditory stimuli to the user;
a visual stimulation device configured to deliver visual light stimuli to the user; and
a closed-loop controller configured to dynamically adjust parameters of the auditory stimuli and the visual stimuli in a synchronized manner based on the determined brain state or sleep stage to influence the user's brainwave activity.

2. The system of claim 1, wherein the auditory stimulation device is configured to generate binaural tones.

3. The system of claim 1, wherein the visual stimulation device comprises one or more light sources configured to emit light at a wavelength selected to avoid substantial melatonin suppression.

4. The system of claim 1, wherein the closed-loop controller is further configured to introduce a stimulation component at approximately 40 Hz into at least one of the auditory stimuli or the visual stimuli during one or more selected sleep stages.

5. The system of claim 1, further comprising at least one electrooculography (EOG) sensor, wherein the processor utilizes signals from the EOG sensor to classify sleep stages.

6. The system of claim 1, further comprising a motion sensor, wherein the processor uses motion data in conjunction with brain activity signals to confirm sleep stage.

7. The system of claim 1, further comprising a machine learning system configured to personalize one or more stimulation parameters based on historical brain activity data and stimulation results for the individual user.

8. The system of claim 1, wherein the system is integrated into a wearable headband that includes the auditory stimulation device and an eye mask housing the visual stimulation device.

9. A method of enhancing sleep quality, comprising:
sensing real-time brain activity of a user;
determining a current brain state or sleep stage based on the sensed brain activity;
delivering auditory stimuli to the user;
delivering synchronized visual light stimuli to the user; and
dynamically modulating, in closed-loop fashion, both the auditory stimuli and the visual light stimuli in response to the determined brain state or sleep stage so as to entrain the user's brainwaves toward frequencies associated with improved sleep quality and neurological health.

10. The method of claim 9, further comprising applying a 40 Hz stimulation component in at least one of the auditory or visual stimuli during detected deep sleep or REM sleep stages.

11. The method of claim 9, further comprising:
logging brain activity data and applied stimulation parameters; and
using machine learning to adapt future stimulation parameters for the specific user.

12. A wearable apparatus for improving sleep, comprising:
a wearable structure configured to be positioned on or near a user's head during sleep;
brain activity sensing means coupled to the wearable structure;
audio delivery means;
light delivery means positioned to provide visual stimulation to the user's eyes while the user is sleeping; and
control means for synchronously adjusting the audio delivery means and the light delivery means in a closed-loop manner based on real-time brain activity measurements.

13. The system of claim 1, wherein the processor determines the brain state or sleep stage by (i) performing a Fast Fourier Transform (FFT) to compute power spectral density (energy density) across frequency bands defined by the American Academy of Sleep Medicine (AASM) and (ii) applying a machine-learning classifier that operates in real time using only data accumulated from the beginning of the sleep session to the current moment.

14. The system of claim 13, wherein the machine-learning classifier is initially trained on publicly available polysomnography datasets containing expert human-scored sleep stages and is further configured to adapt and improve its accuracy for the individual user based on the user's own historical brain-activity and stimulation data collected over multiple sleep sessions.

15. The system of claim 1, wherein the auditory stimulation device comprises comfortable wired earbuds connected by wires to the on-board microprocessor and configured to fit comfortably under the headband.

16. The method of claim 9, wherein the closed-loop modulation comprises four distinct behavioral modes executed in real time based on the determined sleep stage:
(a) Onset Descent mode (Wake→N1→N2) in which the binaural beat frequency leads the current effective brainwave frequency by approximately 2-4 Hz while maintaining a 40 Hz carrier tone, with PWM red-light pulsing synchronized to the beat envelope;
(b) Induction mode (N2→N3) in which the binaural beat target is set to 1-3 Hz and stimulation fades once is confirmed;
(c) Maintenance mode in which stimulation is minimal or absent while is sustained and re-engages upon detection of drift toward lighter stages; and
(d) REM Reinforcement mode in which the binaural beat shifts to the theta band (4-8 Hz) to reinforce endogenous REM theta rhythm while the 40 Hz carrier continues for gamma reinforcement and visual stimulation is softer and ambient.

17. The method of claim 16, wherein the PWM red-light pulsing is synchronized to the binaural beat envelope in Onset Descent and Induction modes, and the visual stimulus becomes softer and ambient in REM Reinforcement mode.

18. The method of claim 16, wherein, upon detection of an arousal event or an undesired stage transition from to a lighter sleep stage prior to achievement of a target slow-wave sleep duration, the closed-loop controller bypasses the normal stage-dispatch decision and directly re-instates the N3 Maintenance mode stimulus parameters without passing through the full mode-dispatch logic.

19. The method of claim 16, wherein the N3 Maintenance mode further comprises:
bandpass filtering the EEG signal in real time at 0.5-1.5 Hz, detecting the positive peak of each slow oscillation cycle corresponding to the cortical up-state, and synchronizing the PWM envelope of the red-light stimulus to the detected up-state phase.

20. The method of claim 16, wherein the REM Reinforcement mode further comprises detecting rapid eye movement (REM) saccade burst events via the EOG sensor and delivering brief, phase-locked LED pulses synchronized to the onset of each detected REM burst.

21. The method of claim 16, wherein, in the Onset Descent mode and N3 Induction mode, the binaural beat frequency is dynamically computed as a leading gradient that is continuously maintained a predetermined offset (approximately 2-4 Hz in Onset Descent and 1-2 Hz in N3 Induction) below the current effective brainwave frequency, converging toward 1 Hz as the brain approaches and enters N3 sleep.
